(19) United States
(12) Patent Application Publication
(10) Pub. No.: US 2021/0308384 A1
(43) Pub. Date: Oct. 7, 2021

(54) SYSTEM AND MEDICAMENT DELIVERY DEVICE CASE

(71) Applicant: SHL MEDICAL AG, ZUG (CH)

(72) Inventors: Yung-Han Wang, Taoyuan City (TW); Chih-Shuen Lin, Taoyuan City (TW)

(21) Appl. No.: 17/264,437

(22) PCT Filed: Sep. 26, 2019
(86) PCT No.: PCT/EP2019/076055

§ 371 Date: Jan. 29, 2021

(30) Foreign Application Priority Data
Oct. 4, 2018 (EP) 18198637.3

(51) Int. Cl. A61M 5/315; A61M 5/00

(52) U.S. Cl. CPC A61M 5/31568; A61M 2205/3546; A61M 5/3158; A61M 5/002

(57) ABSTRACT

The present application pertains to a system for monitoring administration of medicaments from a medicament delivery device, where the system has a case for said medicament delivery device, the case having a holder for holding a medicament delivery device; an image capturing device positioned in relation to said holder so as to capture at least one image of a medicament container in said medicament delivery device; said system also having an electronic circuit to which said image capturing device is connected, said electronic circuit comprising handling elements for handling said captured image.

### CROSS REFERENCE TO RELATED APPLICATIONS

The present application is a U.S. National Phase Application pursuant to 35 U.S.C. § 371 of International Application No. PCT/EP2019/076055 filed Sep. 26, 2019, which claims priority to European Patent Application No. 18198637.3 filed Oct. 4, 2018. The entire disclosure contents of these applications are herewith incorporated by reference into the present application.

### TECHNICAL AREA

The present disclosure relates to a case for a medicament delivery device and in particular a case provided with a number of functions for monitoring the use of the medicament delivery device. It also relates to a system for handling information obtained from the case when a medicament delivery device is placed therein.

### BACKGROUND

The area of self-administering of drugs is expanding and for instance in the medical field of insulin and other medicaments for treating diabetes, medicament delivery devices for self-administration are very common. Regarding for instance diabetes, the patients need to inject e.g. insulin several times per day. In order to be able to handle the medicament delivery devices on a daily basis when working, going to school etc. i.e. travelling to different locations, cases and bags have been developed that users can store their medicament delivery devices, extra needles, medicament containers etc. in. Many of these cases also have cooling elements to keep the insulin below room temperature.

Another aspect of self-medication is that most patients have a treatment scheme set up by their physician that they should follow. However, there are drawbacks and problems connected to this that can cause the patient to miss their treatment. For instance people can forget when they are supposed to medicate. This risk can be reduced by devices that can alert a user when a dose delivery sequence is to be performed. The most simple of these are timers that are triggered by the user. More sophisticated devices can also detect that a dose has been administrated and can calculate a subsequent injection event.

A few of these devices are designed as add-ons that are releasably connected to the medicament delivery device. The drawback with these add-on devices is that there is a further component that the user has to keep track of in addition to the other parts that are needed for the drug administration.

### BRIEF DESCRIPTION OF INVENTION

The aim of the present application is to remedy the drawbacks of the state of the art devices and systems. This aim is solved with a system according to the independent patent claim. Preferable embodiments of the application form the subject of the dependent patent claims.

According to one aspect, a system for monitoring administration of medicaments from a medicament delivery device is provided, where the system comprises a case for the medicament delivery device. The case may comprise a holder for holding a medicament delivery device and an image capturing device positioned in relation to the holder so as to capture at least one image of a medicament container in the medicament delivery device.

The image capturing device may for example comprise a CCD-chip which is a cost-efficient and reliable solution. This may be combined with for example some type of lens, such as a fish-eye lens that can provide a larger image covering area.

The system may comprise an electronic circuit to which the image capturing device is connected, where the electronic circuit may comprise handling elements for handling the captured image. With such a solution, it is possible to obtain information from a medicament delivery device via the image capturing device that does not affect or even touch the medicament delivery device, nor has the medicament delivery device to be modified in order to obtain information. Thus the system will work with any standard medicament delivery device. Preferably the handling elements comprises storage elements for storing said captured image so that the image may later be processed in different ways.

According to one aspect, the image capturing device may be capable of capturing an image comprising the axial position of a stopper inside the medicament container. As an alternative, the image capturing device may be capable of capturing an image comprising the axial position of a piston acting on the medicament container. In this way there is a direct connection between the captured position of a component affecting the dose delivery and a delivered dose. In that regard the electronic circuit may comprise means for comparing the axial position of the stopper or the piston before and after a dose delivery sequence and obtaining the axially moved length of the stopper or the piston.

The information regarding the axial positions of components may be used with information regarding the internal cross-sectional area of the medicament container comprised in the storage elements and wherein the electronic circuit may comprise calculating means for calculating the volume of a delivered dose based on the cross-sectional area and the axially moved length of the stopper or the piston. In order to provide further information, the storage element may comprise information regarding the initial fill volume of the medicament container and wherein the calculating means can calculate remaining volume by subtracting the delivered volume.

According to a favourable solution, the image capturing device may comprise a fisheye lens. With this solution, it is possible to view a component of the medicament container such as the proximal end of the stopper even if it is positioned such that it is obscured by for instance parts of the medicament delivery device when viewed from the side. The fisheye will provide a “curved” image of the medicament container whereby it is possible to provide images that can cover the full stroke length of the stopper in the medicament container.

In order to provide further functionality, the case may further comprise communication elements for transmitting data to and from external data handling units. According to one variant, the calculating means may be comprised in the external data handling units and that the communication elements transmit the captured images, wherein the external data handling units are cloud-based.

As an alternative, the external data handling units may be comprised in a smart device. In this regard, the smart device may be capable of transmitting the processed data to cloud-based data handling resources.

According to another aspect, the calculating means may be comprised in the case and that the communication elements transmit processed data to the external data handling units. In this regard, the communication elements may be capable of transmitting data to a smart device and wherein the smart device is capable of transmitting the processed data to cloud-based data handling resources.

As an alternative, the communication elements may be capable of transmitting the processed data to cloud-based data handling resources.

These and other aspects of, and advantages with, the present disclosure will become apparent from the following detailed description of the present disclosure and from the accompanying drawings.

### BRIEF DESCRIPTION OF DRAWINGS

In the following detailed description of the present disclosure, reference will be made to the accompanying drawings, of which

FIG. 1 shows a schematic view of the interior of a case comprised in the system according to the present application,

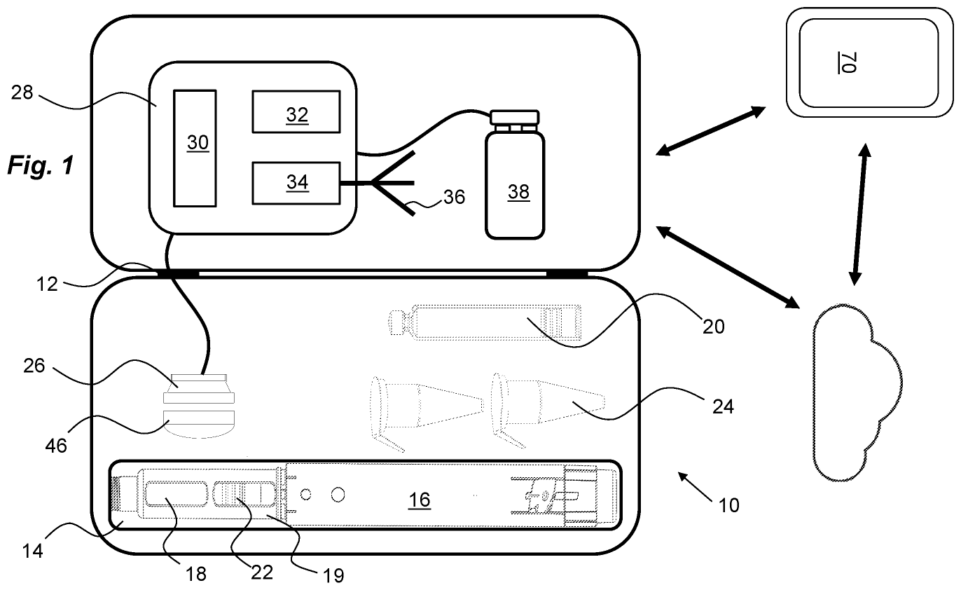

FIG. 2 shows the exterior of the case of FIG. 1,

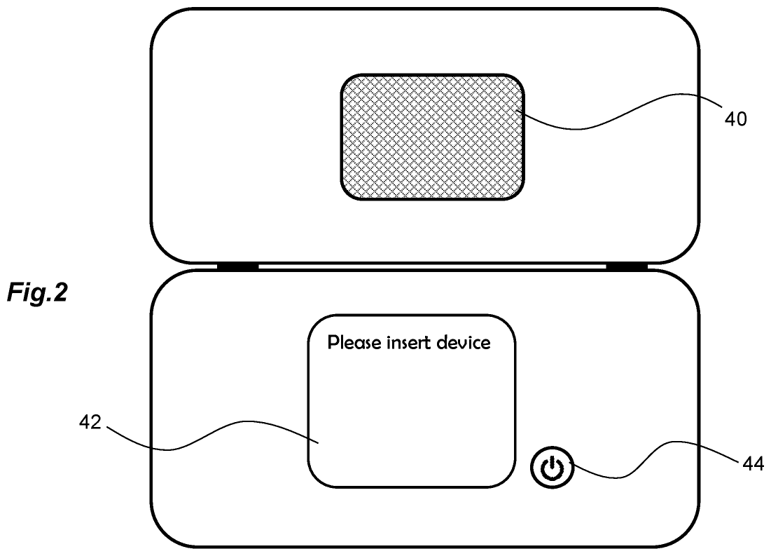

FIG. 3 shows a flow chart of monitoring a medicament delivery device in view of dose delivery,

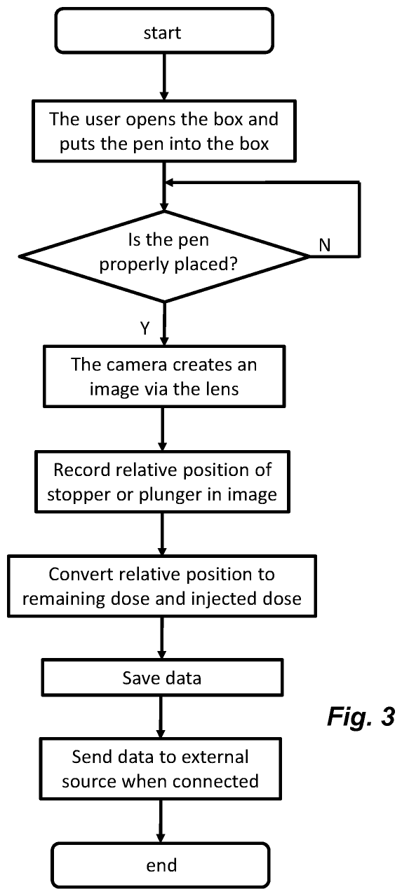

FIGS. 4a, 4b and 4c show different positions of a stopper inside a medicament container where images may be obtained, and

FIGS. 5a, 5b, 6a and 6b show variants of medicament containers that may be used with the present application.

### DETAILED DESCRIPTION

The application shown in the drawings comprises some sort of case or compartment 10 where at least one medicament delivery device can be stored. The case 10 shown is designed with two halves that are connected with hinges 12 or the like connecting elements that can permit opening and closing of the case. A lock or catch (not shown) may further be provided for holding the case closed when not in use. In this regard, it is to be understood that the case 10 may have many different shapes and openings to suit the desired function. The case 10 is in this regard provided with a holder or recess 14 in which a medicament delivery device 16 can be held in a certain predefined position. This is so that information can be obtained as will be described.

The medicament delivery device 16 is provided with a window or opening 18 on its body, for example a medicament container holder 19, through which a medicament container 20 is visible. The medicament container 20 is provided with a stopper 22 that is movable by a drive mechanism of the medicament delivery device 16 for expelling doses of medicament through medicament delivery members 24 such as injection needles. In this regard, the case 10 may contain space for a number of medicament delivery members 24, preferably kept sterile in packages. Moreover, the case 10 may comprise space for additional medicament containers 20 as well as cooling elements for keeping the medicament at storage temperatures.

According to the application, the case 10 is arranged with an image capturing device 26. In the embodiment shown the image capturing device 26 can be a camera or the like. A CCD camera or sensor may for instance be used. The image capturing device 26 is operably connected to an electronic circuit 28 that may comprise handling elements that are designed to handle and process images captures by the image capturing device 26. In this regard, the electronic circuit 28 may comprise a processor 30 and storage elements 32 for storing program code that can operate the image capturing device 26 as well as for storing data from captured images. The electronic circuit 28 may further comprise a communication circuit 34, provided with antennas 36 and transmission circuits for transmitting data from the case to an external data handling unit.

According to one aspect of the communication, the external handling unit may be a smart mobile device 70 wherein the communication circuit may use short-range wireless technologies such as for example ANT+, Bluetooth, NFC, RFID, Wi-Fi, Wireless HART, ZigBee, Z-wave, etc. At present, Bluetooth technology is preferred, being widely spread and included in many so called smart mobile devices, but other communication technologies are naturally feasible.

The mobile devices may then transmit the data from the case 10 to external cloud-based handling resources via communication technologies of the mobile device, such as cellular radio communication networks, e.g. GSM, 3G, 4G, etc. and/or wireless local area networks, which networks can provide access to the internet and thus to a large number of external data storage sources, data handling resources, etc.

Regarding the use of smart mobile devices, it is possible to provide the handling elements for handling the captured images in the smart mobile device 70 instead of in the electronic circuit 28 of the case 10. With this solution, the electronic circuit 28 captures images and then transmits them via the communication circuit 34 for handling by the processors and storage elements of the smart mobile device. The smart mobile device is then arranged with an appropriate program for handling the captured images. Regarding communication technologies, it is of course possible to incorporate the above mentioned communication technologies in the case 10 as such. Then the case 10 will communicate directly with cloud-based data handling resources.

The case 10 further comprises a power source like batteries 38 and/or photovoltaic panels 40. On its outer surface a display 42 may be arranged as well as operation buttons 44 such as on-off button for instance. Instead or in addition to operation buttons, the display 42 may be a touch panel.

As mentioned, the case 10 has a recess or holder 14 that will hold a medicament delivery device 16 in a certain pre-defined position. Moreover, the image capturing device 26 is placed such in relation to the recess or holder 14 that it is capable of obtaining images of the medicament container 20 through the windows 18 of the medicament delivery device 16. In order to provide an optimal image, a lens 46 may be placed in front of the image capturing device 26. For instance the lens 46 may be a so called fish-eye lens. With such a lens 46 it is possible to cover most of the length of the medicament container 20 as will be described.

In operation, a user places a medicament delivery device 16 in the holder 14 of the case 10. Either the electronic circuit of the case 10 is activated by the user opening the case 10 or by placing a medicament delivery device 16 in the case. In that regard, there might be a switch in the holder 14 that is triggered by the medicament delivery device 16. As an alternative, the electronic circuit may be activated by the user pressing the on/off-button 44.

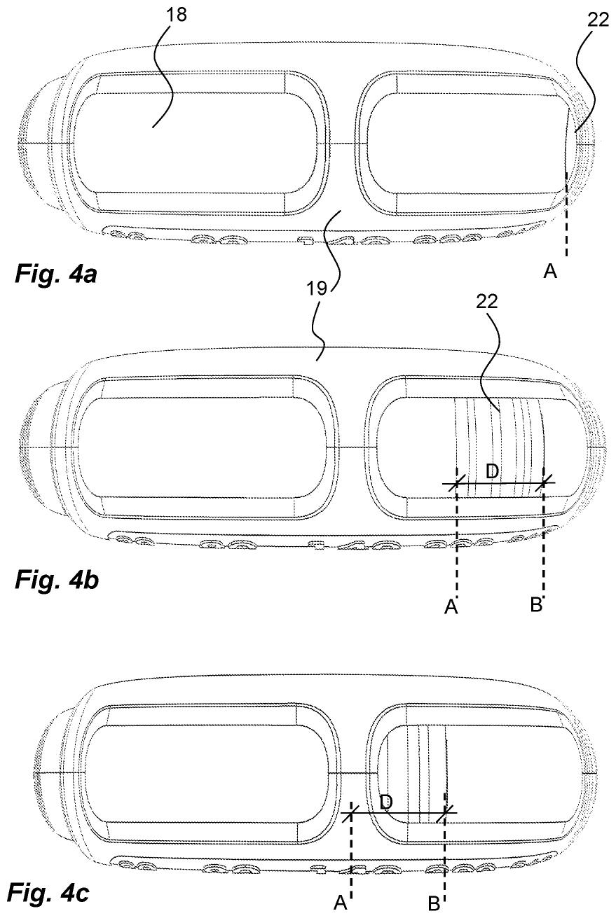

The activation of the electronic circuit 28 will make the image capturing device 26 ready for use. The image capturing device 26 will take an image of the medicament container 20 with its stopper 22 through the openings 18 of the medicament delivery device 16. The handling elements are capable of identifying a proximal end of the stopper 22, as indicated by line A, FIG. 4a, where the position of the proximal end of the stopper 22 inside the medicament container is an indication of the amount of medicament in the medicament container 20. The use of a fisheye lens 46 ensures that the proximal end of the stopper 22 is visible even though it may be obscured by the medicament container holder 19 in its initial position when viewed from the side, providing a curved image of the medicament container 20.

Thus the handling elements are pre-programmed with the initial volume of the medicament container 20. Also, the handling elements are provided with information regarding the inner diameter or cross-sectional area of the medicament container 20. Accordingly when the stopper 22 has moved a certain distance during a medicament delivery sequence and the medicament delivery device 16 is placed in the case 10 and an image is taken, the handling elements can calculate the amount of medicament delivered and also calculate the remaining amount of the medicament container 20.

The handling elements are also capable of handling images where the proximal end of the stopper 22 may be hidden by parts of the medicament container 20. The handling elements may also detect the distal end of the stopper 22 as indicated by line B, FIG. 4b. As seen in FIG. 4b, the handling elements may calculate the axial distance D between line A and line B, and with distance D, the handling elements can calculate the position of line A, i.e. the proximal end of the stopper, even if only the distal end of the stopper 22 is visible at certain positions as shown in FIG. 4c.

It is also possible that the container holder 19 or housing part is transparent wherein the stopper is viewed constantly. Then the electronic circuit only needs to be able of detecting the proximal end of the stopper 22 and thus does not need to calculate the axial dimensions of the stopper 22 as described above.

The information regarding delivered dose volume and remaining dose volume may then be stored in suitable storage elements. Further, the handling elements can document a time-stamp when the image was taken and are capable of calculating when the subsequent dose delivery operation is due. This information may for instance be visible on the display 42. Further, the electronic circuit may be arranged with user alert functions such as a loudspeaker or buzzer that can alert a user when it is time for a dose to be administered. In addition or instead, the display 42 may flash or change colour in order to alert the user.

Since the case 10 is provided with a communication circuit 34, the information regarding administered dose quantity and remaining dose quantity as well as the time stamps may be transmitted to external data storage and handling facilities. For instance, the data may be accessible to the physician of the patient, who is able to monitor the patient's adherence to the treatment scheme in a very convenient way. Also the data may be used for monitoring the patient's supply of medicament in order to renew prescriptions and inform the patient that medicament prescriptions are available.

The communication circuit 34 may as mentioned be of a short range type such that the case can communicate with for instance a smart mobile device 70. The smart mobile device 70 may in turn be provided with an app that is capable of communicating with data storage and handling facilities. On the other hand, the communication circuit 34 of the case may be capable of communicating directly via GSM networks or wireless networks. This solution would exclude the necessity of a smart device for the transfer of data from the case. On the other hand, such communication circuits 34 are generally more expensive than short range circuits and may require subscription.

It is further conceivable that only one image is taken by the image capturing device 26. This might for example be if the only required information is that a dose has been delivered without any further requirements of dose size. The electronic circuit 34 may then be capable of detecting that the stopper 22 has moved or not, i.e. already having information regarding the initial position of the stopper 22.

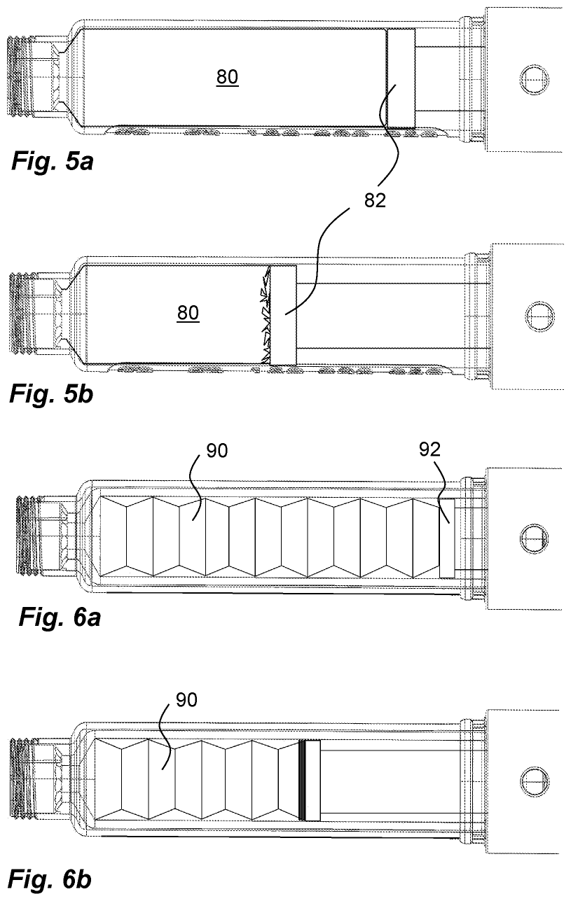

Further, instead of conventional medicament containers 20 having a tubular body with a movable stopper 22, other types of medicament containers may also be employed. FIG. 5a shows a deformable container 80, like a bag that can be deformed so that a dose is delivered. The deformable container 80 may be affected by a plunger or a piston 82 acting on the medicament container such that it collapses during a dose delivery sequence, FIG. 5b.

Another type of medicament delivery device that is feasible is a bellows type medicament container 90 as shown in FIG. 6a. Also here a piston or plunger 92 may act on the distal end of the bellows type medicament container 90 in order to exert a pressure that will deliver a dose of medicament. As the piston is moved forward, the bellows of the bellows type medicament container 90 is compressed and folded as seen in FIG. 6b.

For all these types of medicament containers, the image capturing device 26 is capable of providing an image of the position of the piston 82, 92, for instance its proximal end. As described above, there might only be one image taken, if it is only interesting to detect that a dose has been given or not. If more information is needed or desired, then several images may be captured. The electronic circuit may then be able of calculating the dose volume if it has been pre-programmed with information regarding relationship between incremental movement of the piston 82, 92 and dose quantity. Also the remaining dose may be calculated if the electronic circuit 28 has been pre-programmed with the initial volume of the medicament container 80, 90.

It is to be understood that the embodiment described above and shown in the drawings is to be regarded only as a non-limiting example and that the system may be modified in many ways within the scope of the patent claims.

1-15. (canceled)

16. A system for monitoring administration of medicaments from a medicament delivery device, said system comprising:
a case for said medicament delivery device, the case comprising a holder for holding the medicament delivery device; and
an image capturing device positioned in relation to said holder so as to capture at least one image of a medicament container in said medicament delivery device,
wherein the system further comprises an electronic circuit to which said image capturing device is connected, said electronic circuit comprising handling elements for handling said captured image.

17. The system according to claim 16, wherein said handling elements comprises storage elements for storing said captured image.

18. The system according to claim 16, wherein said image capturing device is capable of capturing an image comprising the axial position of a stopper inside said medicament container or the axial position of a piston acting on said medicament container.

19. The system according to claim 18, wherein said electronic circuit comprises means for comparing the axial position of said stopper or said piston before and after a dose delivery sequence and obtaining the axially moved length of said stopper or said piston.

20. The system according to claim 19, wherein said storage elements comprise information regarding the internal cross-sectional area of said medicament container and wherein said electronic circuit comprises calculating means for calculating the volume of a delivered dose based on the cross-sectional area and the axially moved length of said stopper or said piston.

21. The system according to claim 20, wherein said storage element comprises information regarding the initial fill volume of said medicament container and wherein said calculating means can calculate remaining volume by subtracting the delivered volume.

22. The system according to claim 16, wherein the image capturing device comprises a fisheye lens.

23. The system according to claim 20, wherein said case further comprises communication elements for transmitting data to and from external data handling units.

24. The system according to claim 23, wherein said calculating means is comprised in said external data handling units and that said communication elements transmit said captured images.

25. The system according to claim 24, wherein said external data handling units are cloud-based.

26. The system according to claim 24, wherein said external data handling units are comprised in a smart device.

27. The system according to claim 26, wherein the smart device is capable of transmitting said processed data to cloud-based data handling resources.

28. The system according to claim 27, wherein said calculating means is comprised in said case and that said communication elements transmit processed data to said external data handling units.

29. The system according to claim 28, wherein said communication elements are capable of transmitting data to a smart device and wherein the smart device is capable of transmitting said processed data to cloud-based data handling resources.

30. The system according to claim 28, wherein said communication elements are capable of transmitting said processed data to cloud-based data handling resources.

31. A system for monitoring administration of medicaments from a medicament delivery device, the system comprising:
a case comprising a holder for holding the medicament delivery device;
an image capturing device positioned in relation to the holder so as to capture an image of a medicament container located within in the medicament delivery device when the medicament delivery device is positioned within the holder; and
an electronic circuit connected to the image capturing device, where the electronic circuit comprises components configured to handling said captured image,
wherein the capturing device will record an image comprising an axial position of a stopper inside the medicament container or an axial position of a piston operatively cooperating with the medicament container.

32. The system according to claim 31, the electronic circuit compares the axial position of the stopper or the piston before and after a dose delivery sequence is completed and will record in a storage element an axially moved length of the stopper or the piston.

33. The system according to claim 31 further comprising a storage element that contains information regarding an initial fill volume of the medicament container and a calculating means that will calculate any remaining volume the medicament container by subtracting a measured delivered volume based on axial movement of the stopper or the piston.

34. The system according to claim 31, wherein the image capturing device comprises a fisheye lens.

35. The system according to claim 31 further comprising a communication device that transmits data to a smart device and wherein the smart device transmits processed data collected by the system to a cloud-based data handling resource.
